(19) United States
(12) Patent Application Publication
(10) Pub. No.: US 2014/0011784 A1
(43) Pub. Date: Jan. 9, 2014

(54) METHOD AND COMPOSITION FOR TREATING ASTHMA

(71) Applicant: Jonathan Matz, Columbia, MD (US)

(72) Inventor: Jonathan Matz, Columbia, MD (US)

(21) Appl. No.: 14/023,365

(22) Filed: Sep. 10, 2013

Related U.S. Application Data
(63) Continuation-in-part of application No. 13/298,543, filed on Nov. 17, 2011.

(51) Int. Cl. A61K 31/58; A61M 16/14; A61M 15/00; A61K 45/06; A61M 11/04

(52) U.S. Cl. CPC A61K 31/58; A61K 45/06; A61M 11/04; A61M 15/00; A61M 16/14
USPC 514/171; 128/200.23; 128/203.15; 128/203.12

(57) ABSTRACT

A pharmaceutical composition, method, and article manufacture for the treatment of asthma by inhalation from a pressurized metered dose inhaler (pM DI), a dry powder inhaler (DPI) or nebulizer, the composition and method being based on a combination of a short acting beta agonist (SABA) and an inhaled mometasone in a form adapted to be delivered for inhalation from the MDI, DPI or nebulizer.

### CROSS-REFERENCE TO RELATED APPLICATIONS

This application is a continuation-in-part of U.S. application Ser. No. 13/298,543, filed Nov. 17, 2011 in the U.S. Patent and Trademark Office. All disclosures of the document(s) named above are incorporated herein by reference.

### BACKGROUND OF THE INVENTION

1. Field of the Invention

Aspects of the present invention relate to compositions suitable for the treatment of asthma by inhalation therapy.

2. Description of the Related Art

The entire contents and disclosures of each publication, reference, patent and other prior art referred to herein are incorporated by reference.

Administration via inhalation of long-acting corticosteroids (ICS) in conjunction with long-acting beta-agonists (LABA) has been available for years for the treatment of asthma. For example, the combination of budesonide (an ICS) and formoterol (a LABA) is available under the brand name Symbicort® and is recommended by the National Asthma Education and Prevention Program of the National Institute of Health for long-term control and prevention of symptoms of moderate and severe persistent asthma. The combination is offered in a dry powder inhaler device marketed as ADVAIR Diskus® by GlaxoSmithKline. Budesonide is also marketed worldwide by AstraZeneca under the name Pulmicort®.

Asthma is an inflammatory disease of the lower airways characterized by reversible airway obstruction and bronchial hyper-responsiveness. It is one of the most common diseases in industrialized countries, affecting approximately 130 million people globally. There were 17 million diagnosed asthma sufferers in the USA in 2001.

Asthma is defined by airway inflammation, reversible obstruction and airway hyperresponsiveness. In this disease the inflammatory cells that are involved are predominantly eosinophils, T lymphocytes and mast cells, although neutrophils and macrophages may also be important. Vast arrays of cytokines and chemokines have been shown to be increased in the airways and play a role in the pathophysiology of this disease by promoting inflammation, obstruction and hyperresponsiveness.

Physicians treating asthma have observed that sufferers thereof will use short-acting beta-agonists (SABAs) such as albuterol for relief when their condition flares, often over-using the product when they should also be using in combination therewith, an anti-inflammatory therapy such as an ICS.

However, care givers fear the overuse of albuterol and other SABA's during asthma flare-ups and patients often forget or do not know to use an inhaled steroid if their symptoms deteriorate.

### SUMMARY OF THE INVENTION

Aspects of the subject invention concern a pharmaceutical composition for the treatment of mild, moderate and severe asthma in human patients by inhalation from a pressurized metered dose inhaler (pMDI), a dry powder inhaler (DPI) or a nebulizer, a composition consisting essentially of [1] an inhaled mometasone and [2] a short acting beta agonist (SABA) in a form adapted to be delivered for inhalation from a pMDI, a DPI or a nebulizer at a dose of 5 to 100 mcg of [1] and a dose of 1 to 250 mcg of [2] per delivery.

Another embodiment of the invention comprises a pressurized metered dose inhaler (pMDI), a dry powder inhaler (DPI) or a nebulizer containing the above-described pharmaceutical composition.

### DETAILED DESCRIPTION OF THE EMBODIMENTS

Aspects of the present invention are predicated on the discovery that combinations of a certain inhaled corticosteroid (ICS) and a short acting beta agonist (SABA) offer significant advantages when administered for the treatment of mild, moderate or severe asthma, as well as exercise-induced asthma by inhalation from a pressurized metered dose inhaler (pMDI), a dry powder inhaler (DPI) or a nebulizer not heretofore appreciated by those skilled in the art.

Physicians treating asthma have observed that sufferers thereof will use SABAs for relief when their condition flares, often over-using the product when they should also be using in combination therewith, an anti-inflammatory therapy such as an ICS. Aspects of the present invention are highly advantageous in that it enables an enhanced therapeutic effect in those patients who are poorly compliant with ICS, who overuse SABA during flare-ups of their conditions, and those with mild, moderate or severe asthma that use SABAs on an ‘as needed’ basis.

More particularly, care givers fear the overuse of albuterol and other SABA's during asthma flare-ups and patients often forget or do not know to use an inhaled steroid if their symptoms deteriorate. Aspects of the present invention address these fears by ensuring that patients needing increased SABA for symptoms will be simultaneously receiving anti-inflammatory therapy. Thus, the invention provides the added benefit over and above the use of albuterol and other SABAs alone for prophylactic and recurring use in persistent asthma, intermittent asthma and exercise induced bronchospasm/asthma.

Moreover, it is known that an undesired side-effect (tachyphylaxis) is associated with the prolonged use of LABAs (long acting beta agonist/bronchodilator); however, it has not been shown that the composition of the invention used on an as needed basis gives rise to this highly disadvantageous side-effect.

Utilizing the composition of the invention on an “as needed” basis (2 puffs every 4 hours) will accomplish the goal of asthma symptom control with less overall ICS than if the patient took the ICS product daily as is the current standard practice in the National Heart, Lung, and Blood Institute (NHLBI)—Step 1, Step 2, and Step 3 protocol for treating asthma.

Aspects of the present invention further offer the added benefit of allowing for the on demand use of a combination product without the increased rate of death found in connection with the ICS/LABA (long acting bronchodilators) for use as reliever therapy in asthma exacerbations (O'Byrne—Am J Resp Crit Care Med 2005; 171:129-36 and Rabe—Lancet 2006; 368;744-53). Finally, aspects of the present invention greatly improve patient compliance since only a single payment (or co-pay) is required, thus ensuring anti-inflammatory therapy when the administration of a SABA is required.

Administration may be by inhalation orally or intranasally. The active ingredients are preferably adapted to be administered, either together or individually, from dry powder inhaler(s) (DPIs), especially Turbuhaler®. (Astra AB), pressurized metered dose inhaler(s) (pMDIs), or nebulizer(s). It will be understood by those skilled in the art that the composition may be administered by any suitable such device provided that it is provided with a mechanism operable to actuate the device to deliver the appropriate dose of the medicament of the invention.

Suitable pMDIs for administration of the compositions of the invention are those disclosed in U.S. Pat. Nos. 6,860,262; 7,836,880; and 7,740,463. Pressurized metered-dose inhalers (pM DI) are devices that deliver a specific amount of medication to the luncis, in the form of a short burst of aerosolized medicine that is inhaled by the patient. It is the most commonly used delivery system for treating asthma. Hydrofluoroalkanes (HFA), such as either HFA 134a (1,1,1,2,-tetrafluoroethane) or HFA 227 (1,1,1,2,3,3,3-heptafluoropropane) or combinations of the two are the most commonly used aerosols; however, it will be understood by those skilled in the art that any suitable aerosol may be employed in the practice of the invention.

Suitable DPIs for administration of the compositions of the invention are those disclosed in U.S. Pat. Nos. 7,987,845; 7,958,890; and 7,896,005.

Suitable nebulizers for administration of the compositions of the invention are those disclosed in U.S. Pat. Nos. 6,962,151; 8,015,969; and 8,001,963.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting a compound(s) of the present invention within or to the subject such that it can perform its intended function. Typically, such compounds are carried or transported from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

The pharmaceutical compositions of the invention include a “therapeutically effective amount” or a “prophylactically effective amount” of one or more of the compounds of the invention. A “therapeutically effective amount” refers to an amount effective, at dosages and for periods of time necessary, to achieve the desired therapeutic result, e.g., a diminishment or prevention of effects associated with various disease states or conditions. A therapeutically effective amount of the compound may vary according to factors such as the disease state, age, sex, and weight of the individual, and the ability of the therapeutic compound to elicit a desired response in the individual. A therapeutically effective amount is also one in which any toxic or detrimental effects of the therapeutic agent are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Delivery of the compound of the present invention to the lung by way of inhalation is an important method of treating a variety of respiratory conditions (airway inflammation) noted throughout the specification, including such common local conditions as bronchial asthma and chronic obstructive pulmonary disease. The compound can be administered to the lung in the form of an aerosol of particles of respirable size (less than about 10 pm in diameter). The aerosol formulation can be presented as a liquid or a dry powder. In order to assure proper particle size in a liquid aerosol, as a suspension, particles can be prepared in respirable size and then incorporated into the suspension formulation containing a propellant. Alternatively, formulations can be prepared in solution form in order to avoid the concern for proper particle size in the formulation. Solution formulations should be dispensed in a manner that produces particles or droplets of respirable size.

The following are specific illustrative examples of the invention wherein the total amounts of each product will depend on the size of the canister:

Combination of mometasone furoate [115 or 225 mcg from the valve] with albuterol tartrate [108 mcg from the valve] in the propellant HFA-134a (1,1,1,2 tetrafluoroethane) and ethanol in a pMDI.

Combination of mometasone furoate [115 or 225 mcg from the valve] with levalbuterol tartrate [59 mcg from the valve] in the propellant HFA-134a (1,1,1,2 tetrafluoroethane) and dehydrated Alcohol USP and Oleic Acid NF in a pMDI.

Those skilled in the art will be aware that similar combinations can be made for dry powder inhalation at the ratios noted for the pMDIs but would not include the propellant (HFAI 34a, or HFA 227) or the alcohol, ethanol or oleic acid. Rather, anhydrous lactose may be utilized.

What is claimed is:

1. A pharmaceutical composition for the treatment of asthma by inhalation from a pressurized metered dose inhaler (pMDI), a dry powder inhaler (DPI) or nebulizer, said composition consisting essentially of [1] an inhaled mometasone and [2] a short acting beta agonist (SABA) in a form adapted to be delivered for inhalation from said pMDI. DPI or nebulizer at a dose of 5 to 100 mcg of [1] and a dose of 1 to 250 mcg of [2] per delivery.

2. The pharmaceutical composition of claim 1, wherein said delivery is from a pMDI.

3. The pharmaceutical composition of claim 1, wherein said delivery is from a DPI.

4. The pharmaceutical composition of claim 1, wherein said delivery is from a nebulizer.

5. The pharmaceutical composition of claim 1, wherein said SABA is salbutamol (albuterol), levalbuterol, pirbuterol, terbutaline, epinephrine, a salt thereof with a pharmaceutically acceptable acid or a mixture thereof.

6. The pharmaceutical composition of claim 4, wherein said SABA is salbutamol (albuterol) and said dose is 108 mcg.

7. The pharmaceutical composition of claim 4, wherein said SABA is levalbuterol and said dose is 59 mcg.

8. A method for treating asthma in human patients comprising the administration to a patient in need thereof a therapeutically effective amount of the pharmaceutical composition of claim 1 by inhalation from a pressurized metered dose inhaler (pMDI), a dry powder inhaler (DPI) or a nebulizer.

9. A pressurized metered dose inhaler (pMDI), a dry powder inhaler (DPI) or a nebulizer containing the pharmaceutical composition of claim 1.

10. An article of manufacture comprising packaging material and a pharmaceutical agent contained within said packaging material wherein said packaging material comprises a label which indicates that said pharmaceutical agent can be used for treating a subject suffering from asthma, and wherein said pharmaceutical agent is a composition according to claim 1.

11. An article of manufacture according to claim 10 wherein said packaging material is a pressurized metered dose inhaler (pMDI).

12. An article of manufacture according to claim 10 wherein said packaging material is a dry powder inhaler (DPI).

13. An article of manufacture according to claim 10 wherein said packaging material is a nebulizer.
